(19) United States
(12) Patent Application Publication
(10) Pub. No.: US 2026/0274830 A1
(43) Pub. Date: Sep. 17, 2026

(54) DEUTERIUM-SUBSTITUTED PYRIDAZINE BENZOTHIOPHENE COMPOUND AND USE THEREOF

(71) Applicant: MEDSHINE DISCOVERY INC., Jiangsu (CN)

(72) Inventors: Haiying HE, Shanghai (CN); Lingyun WU, Shanghai (CN); Lele ZHAO, Shanghai (CN); Jianjun SUN, Shanghai (CN); Shuhui CHEN, Shanghai (CN)

(21) Appl. No.: 18/992,580

(22) PCT Filed: Jul. 14, 2023
(86) PCT No.: PCT/CN2023/107380

§ 371 Date: Feb. 25, 2025

(30) Foreign Application Priority Data
Jul. 14, 2022 (CN) 202210834769.8
Aug. 5, 2022 (CN) 202210936980.0

(51) Int. Cl. C07D 409/14; A61K 31/501; C07B 59/00

(52) U.S. Cl. CPC C07D 409/14; A61K 31/501; C07B 59/002

(57) ABSTRACT

The present invention relates to a class of deuterium-substituted pyridazine benzothiophene compounds and the use thereof.

The present application claims the right of the following priorities:
CN202210834769.8, Jul. 14, 2022;
CN202210936980.0, Aug. 5, 2022.

### TECHNICAL FIELD

The present disclosure relates to a class of deuterium-substituted pyridazine benzothiophene compounds and uses thereof.

### BACKGROUND

The NLRP3 inflammasome is a multiprotein complex that plays a crucial role in innate immunity and the development of inflammation-related diseases. The NLRP3 inflammasome is composed of NOD-like receptors (NLRs), apoptosis-associated speck-like protein containing a CARD (ASC), and caspase-1. NLRP3 can be activated by exogenous pathogens or endogenous risk factors such as mitochondrial reactive oxygen species, oxidized mitochondrial DNA, β-amyloid, or α-synuclein. Activated NLRP3 assembles with ASC and caspase-1 to form the activated NLRP3 inflammasome, which further hydrolyzes IL-1ß precursor (pro-IL-1β) and IL-18 precursor (pro-IL-18) via caspase-1, leading to the release of the active cytokines IL-1β and IL-18. The secretion of these cytokines can lead to pyroptosis. The NLRP3 inflammasome plays an important role in the development of various autoimmune diseases, cardiovascular diseases, neurodegenerative diseases, and tumor (Nature Reviews Drug Discovery, 2018, 17 (8): 588-606).

There are currently no drug molecules for NLRP3 inhibitors on the market, and drugs such as OLT-1177, Inzomelid, and IFM-2427 are in the clinical research stage. The development of NLRP3 inhibitors has broad application prospects.

There are currently no drug molecules for NLRP3 inhibitors on the market. The preclinical compound MCC950 has a significant inhibitory effect on NLRP3 (Sci. Transl. Med. 10, eaah4066 (2018)). Drugs such as OLT-1177, Inzomelid, and IFM-2427 are in the clinical research stage. The development of NLRP3 inhibitors has broad application prospects.

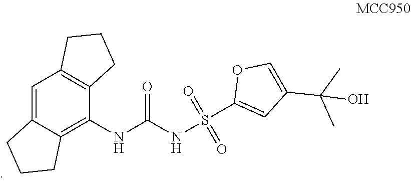 [SMILES: CC(C)(O)c1coc(S(=O)(=O)NC(=O)Nc2c3c(cc4c2CCC4)CCC3)c1]

### CONTENT OF THE PRESENT INVENTION

The present disclosure provides a compound of the following formula, a stereoisomer thereof, or a pharmaceutically acceptable salt thereof,

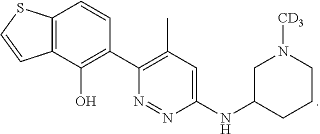 [SMILES: [2H]C([2H])([2H])N1CCCC(Nc2cc(C)c(-c3ccc4sccc4c3O)nn2)C1]

In some embodiments of the present disclosure, the compound, the stereoisomer thereof, or the pharmaceutically acceptable salt thereof is provided, and the compound is selected from:

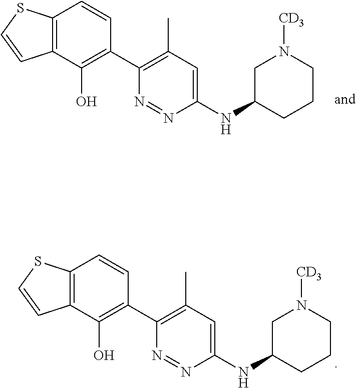 [SMILES: [2H]C([2H])([2H])N1CCC[C@@H](Nc2cc(C)c(-c3ccc4sccc4c3O)nn2)C1 ; [2H]C([2H])([2H])N1CCC[C@@H](Nc2cc(C)c(-c3ccc4sccc4c3O)nn2)C1]

The present disclosure further provides a use of the compound, the stereoisomer thereof, or the pharmaceutically acceptable salt thereof in the manufacture of a medicament for treating Parkinson's disease.

The present disclosure further provides a method for treating Parkinson's disease in a subject in need thereof, comprising administering to the subject an effective amount of the compound, the stereoisomer thereof, or the pharmaceutically acceptable salt thereof.

The present disclosure further provides the following synthesis methods:

### Synthesis of Intermediate AA

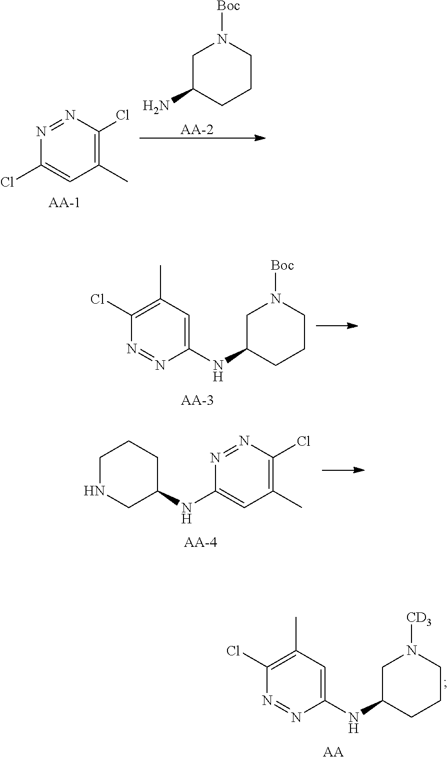

### Synthesis of Intermediate BB

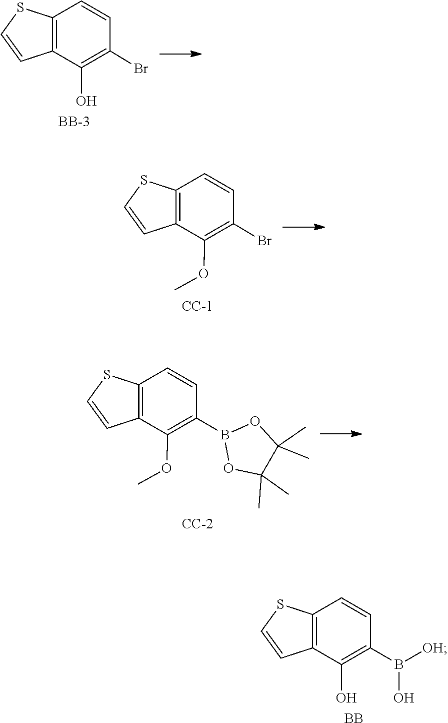

### Synthesis of Compound II

### Method 1:

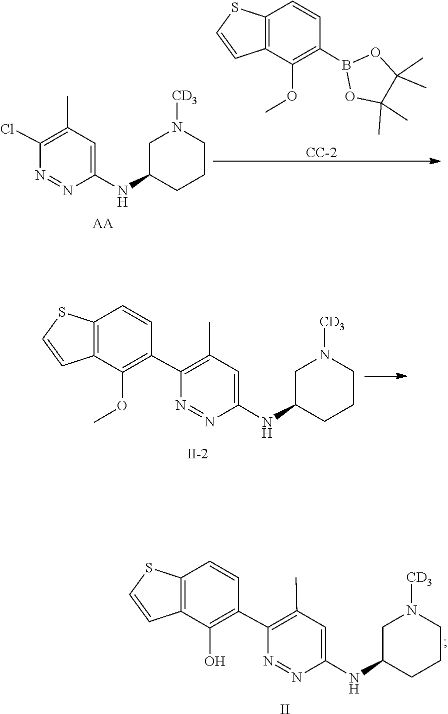

### Method 2:

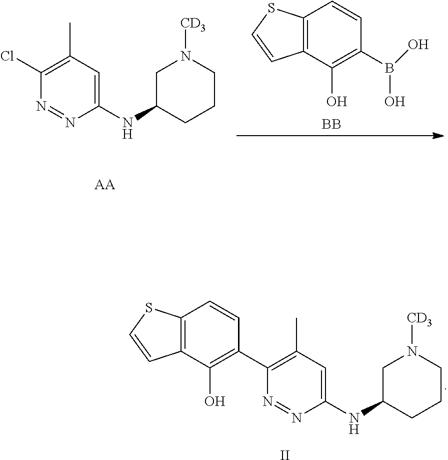

The present disclosure further provides the following experimental test methods for the above-mentioned compound or the pharmaceutically acceptable salt thereof:

### Test Method 1: Pharmacokinetic Evaluation of Compound in CD-1 Mice In Vivo

Experimental objective: To test the pharmacokinetics of compound in CD-1 mice

### Experimental Materials:

CD-1 mice (male, 7 to 9 weeks old)

### Experimental Operation:

The pharmacokinetic characteristics of the compound in rodents after intravenous injection and oral administration were tested according to the standard protocol. In the experiment, the candidate compound was formulated into clear solutions, and a single intravenous injection and oral administration were given to mice. The vehicle for intravenous injection and oral administration was a mixed vehicle composed of 5% dimethyl sulfoxide and 95% solutol with a content of 5%. Four male CD-1 mice were used in this project. Two mice were administered via intravenous (IV) injections, and plasma samples were collected at 0 h (pre-administration) and 0.0833, 0.25, 0.5, 1, 2, 4, 8, and 24 h post-administration. The other two mice were administered orally by gavage (PO), and plasma samples were collected at 0 h (pre-administration) and 0.25, 0.5, 1, 2, 4, 8, and 24 h post-administration. Plasma concentrations were quantitatively analyzed by LC-MS/MS analysis method, and pharmacokinetic parameters such as peak concentration (Cₘₐₓ), clearance (CL), half-life (T_{1/2}), volume of distribution at steady state (Vdss), area under the drug-time curve (AUC₀₋ₗₐₛₜ), and bioavailability (F) were calculated.

Conclusion: The compound of the present disclosure has good pharmacokinetic properties in CD-1 mice in vivo, including good oral bioavailability, oral exposure, half-life, clearance, etc.

### Test Method 2: Pharmacokinetic Evaluation of Compounds in SD Rats In Vivo

Experimental objective: To test the pharmacokinetics of compound in SD rats in vivo

### Experimental Materials:

SD rat (male, 150 to 180 g)

### Experimental Operation:

The pharmacokinetic characteristics of the compound in rodents after intravenous injection and oral administration were tested according to the standard protocol. In the experiment, the candidate compound was formulated into clear solutions, and a single intravenous injection and oral administration were given to SD rats. The vehicle for intravenous injection and oral administration was a mixed vehicle composed of 5% dimethyl sulfoxide and 95% solutol with a content of 5%. Four male SD rats were used in this project. Two SD rats were administered via intravenous injections, and plasma samples were collected at 0.083, 0.25, 0.5, 1, 2, 4, 8, and 24 h post-administration. The other two SD rats were administered orally by gavage, and plasma samples were collected at 0.25, 0.5, 1, 2, 4, 8, and 24 h post-administration. Plasma concentrations were quantitatively analyzed by LC-MS/MS analysis method, and pharmacokinetic parameters such as peak concentration (Cₘₐₓ), clearance (CL), half-life (T_{1/2}), volume of distribution at steady state (Vdss), area under the drug-time curve (AUC₀₋ₗₐₛₜ), and bioavailability (F) were calculated.

Conclusion: The compound of the present disclosure has good pharmacokinetic properties in SD rats in vivo, including good oral bioavailability, oral exposure, half-life, clearance, etc.

Test Method 3: Inhibition Test of Compounds on hERG Potassium Ion Channels

Experimental objective: To detect the effect of the test compounds on hERG potassium ion channels using a fully automated patch-clamp method.

### Experimental Method

### 1. Cell Culture

1.1 CHO-hERG cells were cultured in a 175 cm² culture flask. When the cell density reached 60-80%, the culture medium was removed, and the cells were washed once with 7 mL of PBS (Phosphate Buffered Saline). Then, 3 mL of digestion solution was added for digestion.

1.2 After complete digestion, 7 mL of culture medium was added to neutralize the solution. The mixture was then centrifuged, the supernatant was removed, and the cells were resuspended in 5 mL of culture medium to ensure a cell density of 2-5×10⁶/mL.

### 2. Solution Preparation is Shown in Table 1:

TABLE 1
Composition of intracellular and extracellular solutions

| | Extracellular | Intracellular |
|---|---|---|
| | solution | solution |
| Reagent | (mM) | (mM) |
| CaCl₂ | 1 | 1 |
| MgCl₂ | 1.25 | 1 |
| KCl | 5 | 140 |
| NaCl | 140 | — |
| Glucose | 10 | — |
| 4- | 10 | 10 |
| Hydroxyethylpiperazineethanesulfonic | | |
| acid buffer solution | | |
| Ethylene glycol tetraacetic acid | — | 10 |
| chelating agent | | |
| pH | Adjusted pH | Adjusted pH |
| | with NaOH | with KOH |
| | to 7.4 | to 7.4 |

Note:
“—” indicates the absence of the reagent.

### 3. Electrophysiological Recording Process

The processes of single-cell high-impedance sealing and whole-cell mode formation were all automatically completed by the Qpatch instrument. After obtaining the whole-cell recording mode, the cells were clamped at −80 mV. Before a 5-second +40 mV depolarization stimulus was applied, a 50-millisecond pre-voltage of −50 mV was given, followed by repolarization to −50 mV for 5 seconds, and then returned to −80 mV. This voltage stimulus was applied every 15 seconds. After recording for 2 minutes, extracellular solution was given and recorded for 5 minutes. Then the drug administration process began, starting with the lowest test concentration of the compound, each concentration was applied for 2.5 minutes, and after all concentrations were applied consecutively, a positive control compound, 3 μM Cisapride, was given. At least 3 cells were tested for each concentration (n≥3).

### 4. Compound Preparation

4.1 The compound stock solution was diluted with DMSO. 10 μL of the compound stock solution was added to 20 μL of DMSO solution and then sequentially 3-fold diluted to obtain 6 different DMSO concentrations.

4.2 4 μL of each of the 6 DMSO concentrations of the compound was taken and added to 396 μL of extracellular solution, 100-fold diluted to obtain 6 intermediate concentrations. Then, 80 μL of each of the 6 intermediate concentrations of the compound was taken and added to 320 μL of extracellular solution, 5-fold diluted to obtain the final test concentrations.

4.3 The highest test concentration was 40.00 μM, followed by concentrations of 40.00, 13.33, 4.44, 1.48, 0.49, and 0.16 μM, totaling 6 concentrations.

4.4 The DMSO content in the final test concentration did not exceed 0.2%, and this concentration of DMSO had no effect on hERG potassium channels.

4.5 The entire dilution process for the compound preparation was carried out by the Bravo instrument.

### 5. Data Analysis

Experimental data were analyzed using GraphPad Prism 5.0 software.

Conclusion: The compounds of the present disclosure exhibit no significant inhibitory effect on hERG potassium channels.

### Test Method 4: Behavioral and Histological Testing of 6-Hydroxydopamine-Induced Parkinson's Disease Model in Rats

### I. Experimental Objective:

The Parkinson's disease (PD) model was induced in rats by unilateral administration of 6-hydroxydopamine (6—OHDA) to two brain regions of substantia nigra (SN) and striatum (Str), followed by corresponding behavioral tests (apomorphine-asymmetric rotation test, balance beam test, rotarod test). Some animals were selected for histological evaluation (tyrosine hydroxylase (TH), microglia (Iba-1) immunofluorescence staining, and Western blot sampling, as well as neurotransmitter detection sampling).

### II. Experimental Subjects and Grouping:

69 male SD rats (180 to 220 g), with the specific dosing regimen shown in Table 2.

TABLE 2
Detailed list of dosing regimens for experimental subjects

| | | | | Number | | |
|---|---|---|---|---|---|---|
| | | Administration | | of | Route of | Administration |
| Group | Animal | group | Dose | animals | administration | time point |
| 1 | SD rat | Sham surgery | / | 12 | / | / |
| | | group | | | | |
| 2 | SD rat | 6-OHDA model | / | 12 | / | / |
| | | group | | | | |
| 3 | SD rat | 6-OHDA + | 5 | 15 | p.o. | Twice daily/21 |
| | | compound | mpk | | | consecutive |
| | | | | | | days post- |
| | | | | | | modeling |
| 4 | SD rat | 6-OHDA + | 5 | 15 | p.o. | Twice daily/21 |
| | | compound | mpk | | | consecutive |
| | | | | | | days post- |
| | | | | | | modeling |

### III. Experimental Methods:

### 1. Stereotaxic Drug Administration in SN and Str Regions (Unilateral Drug Administration, Right Side)

SN: AP=−5.0 mm; ML=+1.9 mm; DV=−8.5 mm;
Str: AP=+0.5 mm; ML=+3.0 mm; DV=−6.0 mm.

Note: AP is the anterior-posterior distance from the bregma (Y-axis), ML is the medial-lateral distance from the sagittal suture (X-axis), and DV is the vertical downward distance from the cranial surface (Z-axis).

6—OHDA (20 μg/8 μL) was dissolved in 0.9% normal saline (NS) (containing 0.02% ascorbic acid) and administered at a rate of 0.4 μL/min for 10 minutes. Each animal was given 4 μL in both the SN and Str regions. The Sham group was given 0.9% NS (containing 0.02% ascorbic acid). The details were as follows:
1) Animal weighing and anesthesia: The animals were anesthetized.
2) Rat head fixation: The animal's head was fixed to ensure no movement and the brain surface was leveled.
3) Determination of the bregma: The rat's head was shaved, and a skin incision was made along the sagittal suture to expose the bregma.
4) Positioning of SN and Str regions: A glass electrode was positioned at the bregma, the axes on the coordinate display were set to zero, and the positioning was done based on the coordinates.
5) Injection: After slowly inserting the needle to the SN and Str regions and waiting for 10 minutes, the drug was administered at a rate of 0.4 μL/min. After administration, the needle was left in place for another 10 minutes before being slowly withdrawn.

### 2. Apomorphine-Asymmetric Rotation Test

1) Drug Injection: Apomorphine (0.5 mg/kg) was administered intraperitoneally.
2) Acclimation: Before testing, the animals were placed in the test room to acclimate for 30-60 minutes.
3) Experimental method: Apomorphine (APO) was administered intraperitoneally (0.5 mg/kg) to observe behavioral changes.
4) Result analysis: When apomorphine induced a positive rotational behavior, the rats predominantly used the forelimb on the rotating side as a support point to rotate in situ towards the contralateral side of the lesion. The rotational behavior of the animals was monitored for 30 minutes, with a frequency of more than 5 rotations per minute being considered a quantitative indicator of successful PD modeling.

### 3. Balance Beam Test

1) Two days before the experiment, the mice were placed on the balance beam to acclimate for 10 minutes each day, with each training session involving crossing the balance beam twice. Typically, the mice crossed the beam with minimal pauses. When the mice stopped to sniff or look around without moving forward, the experimenter should wear gloves to prod or push them from behind to encourage forward movement.
2) After the experiment began, the mice were placed on the balance beam, and the time taken to cross the beam and the number of slips were recorded.
3) After each animal completed the experiment, feces were removed, and the balance beam was sprayed with 75% alcohol and wiped dry with clean gauze.
4) Evaluation criteria: The average time of two successful crossings of the balance beam and the number of slips (when a paw left the top of the balance beam).

### 4. Rotarod Test (Muscle Strength Test)

1) Acclimation: Before testing, the animals were placed in the test room to acclimate for 30-60 minutes.
2) Acclimation training: Each experimental animal was placed on the rotarod fatigue apparatus for acclimation training for 5 minutes.
3) Formal testing: The rotarod fatigue apparatus was set to a speed of 20 rpm/min and a test time of 5 minutes. The mice were placed on the rotarod in batches for testing. After each round, feces and urine had to be removed, and the rotarod was disinfected with 75% alcohol and wiped dry.

Result analysis: The time each animal stayed on the rod was counted.

### 5. Immunofluorescence Staining (TH, Iba-1)

1) Animal weighing and anesthesia: The animals were anesthetized.
2) Mouse fixation and perfusion: The mice were fixed on a dissection board, their chest cavities were opened, the right atrial appendage was cut, and NS was perfused into the left ventricle at a rate of 30 rpm/min. After the blood was flushed out, 4% paraformaldehyde (PFA) was perfused to fix the tissue, and then the intact brain tissue was peeled off.
3) Brain fixation and sucrose immersion: The extracted brain tissue was immersed in 4% PFA and stored in a 4° C. refrigerator overnight. The brain tissue was then transferred to 20%, 30%, and 35% gradient sucrose solutions for sucrose immersion (the time or concentration of the sucrose solution was increased as necessary based on the sucrose immersion status of the brain tissue).
4) The brain tissue was removed and embedded in OCT embedding agent, and sectioned using a cryostat at a thickness of 16 μm. After sectioning, the sections were stored at −20° C. or −80° C.
5) The sections were rewarmed for 30 minutes.
6) Blocking: 10% serum+0.3% TritonX-100 at room temperature for 1 hour.
7) After cytospinning, the sections were added with the primary antibody and incubated overnight at 4° C.
8) The sections were rewarmed for 30 minutes.
9) The sections were washed to remove excess primary antibody for 5 minutes, 3 times.
10) The sections were incubated with the secondary antibody under backlight for 2 hours at room temperature.
11) The sections were washed to remove excess secondary antibody for 5 minutes, 3 times.
12) 4′,6-Diamidino-2-phenylindole (DAPI) was added at room temperature for 10 minutes.
13) The sections were washed to remove excess DAPI for 5 minutes, 3 times.
14) Mounting: glycerol (70%), avoiding air bubbles.

Conclusion: The compounds of the present disclosure have the effect of improving behavioral indicators and enhancing the expression of TH in the striatum compared to the 6-hydroxydopamine model group.

### Technical Effect

The NLRP3 inhibitor provided by the present disclosure can effectively inhibit the activity of NLRP3 and the activation of downstream caspase-1, thereby inhibiting the maturation and secretion of IL-1B.

### Definition and Description

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting such compounds with a sufficient amount of base in a pure solution or a suitable inert solvent. When the compound of the present disclosure contains a relatively basic functional group, an acid addition salt can be obtained by contacting the compound with a sufficient amount of acid in a pure solution or a suitable inert solvent. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and(S)-enantiomers, diastereoisomers, (D)-isomers, (L)-isomers, racemic, and other mixtures thereof, such as enantiomers or diastereomer enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) r a straight dashed bond ( [symbol] ).

The compounds of the present disclosure may exist in specific forms.

Unless otherwise specified, the term “tautomer” or “tautomeric form” means that at room temperature, the isomers of different functional groups are in dynamic equilibrium and can be transformed into each other quickly. If tautomers possibly exist (such as in solution), the chemical equilibrium of tautomers can be reached. For example, proton tautomer (also called prototropic tautomer) includes interconversion through proton migration, such as keto-enol isomerization and imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one.

Unless otherwise specified, the terms “enriched in one isomer”, “enriched in isomers”, “enriched in one enantiomer”, or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and(S)-isomers, or D and L isomers can be prepared using chiral synthesis, chiral reagents, or other conventional techniques. If one kind of enantiomer of certain compound of the present disclosure is to be obtained, it can be obtained by asymmetric synthesis or derivative action of chiral auxiliary, wherein the resulting diastereomeric mixture is separated and the auxiliary group is cleaved to provide the pure desired enantiomer. Alternatively, when the molecule contains a basic functional group (such as amino) or an acidic functional group (such as carboxyl), a salt of a diastereoisomer is formed with an appropriate optically active acid or base, and then diastereomeric resolution is performed by conventional methods known in the art, and then the pure enantiomer is recovered. In addition, the enantiomer and the diastereoisomer are generally separated through chromatography which uses a chiral stationary phase and optionally combines with a chemical derivative method (such as carbamate generated from amine).

The compound of the present disclosure may contain an unnatural proportion of atomic isotope at one or more than one atom that constitutes the compound. For example, the compound can be radiolabeled with a radioactive isotope, such as tritium (³H), iodine-125 (¹²⁵I), or C-14 (¹⁴C). For another example, deuterated drugs can be formed by replacing hydrogen with deuterium, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequent event or condition may occur but not requisite, that the term includes the instance in which the event or condition occurs and the instance in which the event or condition does not occur.

The term “substituted” means one or more than one hydrogen atom on a specific atom is substituted by the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone.

The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted by 0 to −2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when L in A-L-Z represents a single bond, the structure of A-L-Z is actually A-Z.

Unless otherwise specified, when a group has one or more than one linkable site, any one or more than one site of the group can be linked to other groups through chemical bonds. When the linking site of the chemical bond is not positioned, and there is an H atom at the linkable site, then the number of H atoms at the site will decrease correspondingly with the number of the chemical bonds linking thereto so as to meet the corresponding valence. The chemical bond between the site and other groups can be represented by a straight solid bond ( [symbol] ) a straight dashed bond ( [symbol] ) or a wavy line

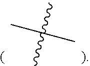

For example, the straight solid bond in —OCH₃ means that it is linked to other groups through the oxygen atom in the group; the straight dashed bonds in

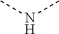

means that it is linked to other groups through the two ends of nitrogen atom in the group; the wave lines in

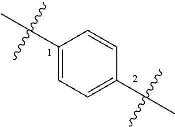 [SMILES: *c1ccc(*)cc1]

means that the phenyl group is linked to other groups through carbon atoms at position 1 and position 2.

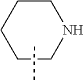

means that it can be linked to other groups through any linkable sites on the piperidinyl by one chemical bond, including at least four types of linkage, including

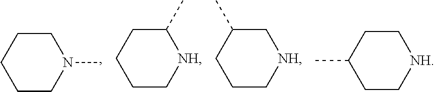

Even though the H atom is drawn on the —N—,

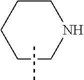

still includes the linkage of

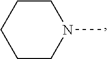

merely when one chemical bond was connected, the H of this site will be reduced by one to the corresponding monovalent piperidinyl.

The term “leaving group” refers to a functional group or atom which can be replaced by another functional group or atom through a substitution reaction (such as nucleophilic substitution reaction). For example, representative leaving groups include triflate; chlorine, bromine, and iodine; sulfonate group, such as mesylate, tosylate, p-bromobenzenesulfonate, p-toluenesulfonate; acyloxy, such as acetoxy, trifluoroacetoxy.

The term “protecting group” includes, but is not limited to, “amino protecting group”, “hydroxyl protecting group”, or “mercapto protecting group”. The term “amino protecting group” refers to a protecting group suitable for preventing the side reactions occurring at the nitrogen of an amino. Representative amino protecting groups include, but are not limited to: formyl; acyl, such as alkanoyl (e.g., acetyl, trichloroacetyl, or trifluoroacetyl); alkoxycarbonyl, such as tert-butoxycarbonyl (Boc); arylmethoxycarbonyl such as benzyloxycarbonyl (Cbz) and 9-fluorenylmethoxycarbonyl (Fmoc); arylmethyl, such as benzyl (Bn), trityl (Tr), 1,1-bis-(4′-methoxyphenyl)methyl; silyl, such as trimethylsilyl (TMS) and tert-butyldimethylsilyl (TBS). The term “hydroxyl protecting group” refers to a protecting group suitable for blocking the side reaction on hydroxyl. Representative hydroxyl protecting groups include, but are not limited to: alkyl, such as methyl, ethyl, and tert-butyl; acyl, such as alkanoyl (e.g., acetyl); arylmethyl, such as benzyl (Bn), p-methoxybenzyl (PMB), 9-fluorenylmethyl (Fm), and diphenylmethyl (benzhydryl, DPM); silyl, such as trimethylsilyl (TMS) and tert-butyl dimethyl silyl (TBS).

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, and preferred embodiments include, but are not limited to, the examples of the present disclosure.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), diffraction intensity data are collected from the cultured single crystal using a Bruker D8 venture diffractometer with CuKα radiation as the light source and scanning mode: φ/ω scan, and after collecting the relevant data, the crystal structure is further analyzed by direct method (Shelxs97), so that the absolute configuration can be confirmed.

The solvents used in the present disclosure are commercially available.

The present disclosure adopts the following abbreviations: Alloc stands for allyloxycarbonyl; SEM stands for trimethylsilylethoxymethyl; OTs stands for 4-toluenesulfonyl; Boc stands for tert-butoxycarbonyl; DCM stands for dichloromethane; DIEA stands for N,N-diisopropylethylamine; Mel stands for iodomethane; PE stands for petroleum ether; EA stands for ethyl acetate; THF stands for tetrahydrofuran; EtOH stands for ethanol; MeOH stands for methanol; Boc₂O stands for di-tert-butyl dicarbonate; NH₄Cl stands for ammonium chloride; T₃P stands for propylphosphonic anhydride; Pd/C stands for palladium/carbon catalyst; TMSN₃ stands for azidotrimethylsilane; NCS stands for N-chlorosuccinimide; HBr stands for hydrobromic acid; AcOH stands for acetic acid; HATU stands for 2-(7-azabenzotriazol-1-yl)-N,N,N′,N′-tetramethyluronium hexafluorophosphate; DBU stands for 1,8-diazabicyclo[5.4.0]undec-7-ene; FA stands for formic acid; ACN stands for acetonitrile; TLC stands for thin-layer chromatography; HPLC stands for high performance liquid chromatography; pre-HPLC stands for preparative high performance liquid chromatography; LCMS stands for liquid chromatography-mass spectrometry. DMSO stands for dimethyl sulfoxide; DMSO-d₆ stands for deuterated dimethyl sulfoxide; CD₃OD stands for deuterated methanol; CDCl₃ stands for deuterated chloroform; D₂O stands for deuterium oxide; solutol stands for polyethylene glycol-15-hydroxystearate.

The compounds of the present disclosure are named according to the conventional naming principles in the art or by ChemDraw® software, and the commercially available compounds use the supplier catalog names.

### DETAILED DESCRIPTION OF THE PREFERRED EMBODIMENT

The present disclosure is described in detail by the examples below, but it does not mean that there are any adverse restrictions on the present disclosure. The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, and preferred embodiments include, but are not limited to, the examples of the present disclosure. It will be apparent to those skilled in the art that various variations and improvements can be made to specific embodiments of the present disclosure without departing from the spirit and scope of the present disclosure.

### Example 1: Preparation of Intermediate AA

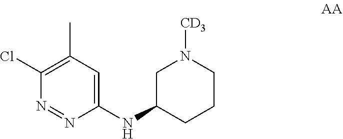 [SMILES: [2H]C([2H])([2H])N1CCC[C@@H](Nc2cc(C)c(Cl)nn2)C1]

### Synthetic Route:

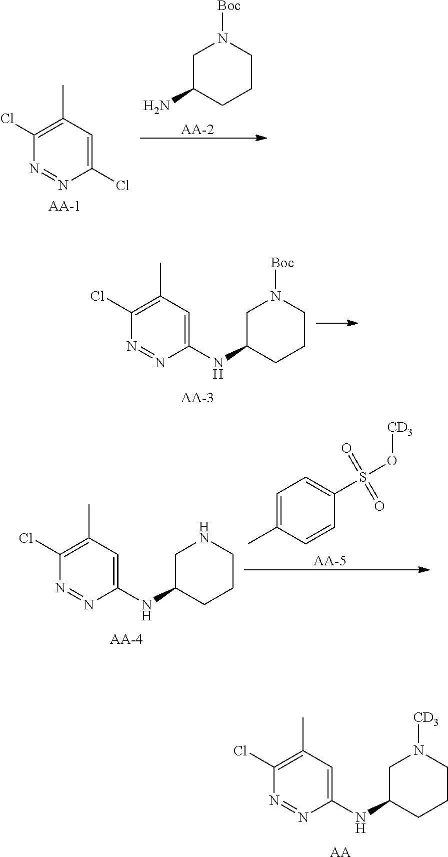

### Step 1

Compound AA-1 (150 g, 920.22 mmol), compound AA-2 (368.60 g, 1.84 mol) and N,N-diisopropylethylamine pyridine (237.86 g, 1.84 mol) were dissolved in N-methylpyrrolidone (1500 mL). Under nitrogen atmosphere, the reaction mixture was stirred at 120° C. for 20 hours. The reaction mixture was added with water (4000 mL) and extracted with ethyl acetate (1000 mL×3). The organic phases were combined, sequentially washed with citric acid (1%, 1000 mL×2) and saturated brine (1000 mL×2), dried over anhydrous sodium sulfate, filtered, and the filtrate was concentrated under reduced pressure. The residue was purified by silica gel column chromatography (eluent: petroleum ether/ethyl acetate=3/1 to 1/1) to obtain compound AA-3. ¹H NMR (400 MHz, CDCl₃) δ 6.55 (s, 1H), 4.74 (br s, 1H), 3.89-3.87 (m, 1H), 3.73-3.69 (m, 1H), 3.49-3.44 (m, 1H), 3.35-3.33 (m, 2H), 2.53 (s, 3H), 1.98-1.95 (m, 1H), 1.75-1.69 (m, 2H), 1.62-1.57 (m, 1H), 1.44 (s, 9H). MS-ESI calculated for [M+H]⁺327, found 327.

### Step 2

Compound AA-3 (53 g, 162.17 mmol) was dissolved in ethyl acetate (500 mL), and a solution of hydrochloric acid in ethyl acetate (4 mol/L, 162 mL) was added to the reaction mixture. The mixture was stirred at 25° C. for 12 hours. The reaction mixture was filtered. The filter cake was washed with ethyl acetate (500 mL) and dried under vacuum to obtain hydrochloride of compound AA-4. MS-ESI calculated for [M+H]⁺227, found 227.

### Step 3

The hydrochloride of compound AA-4 (1.1 g, 4.85 mmol) and triethylamine (981.96 mg, 9.70 mmol) were dissolved in acetonitrile (10 mL). The reaction mixture was stirred at 0° C. for 30 minutes. Then compound AA-5 (1.38 g, 7.27 mmol) was added. The reaction mixture was slowly warmed to 25° C. and stirred for 1.5 hours. The reaction mixture was added with water (10 mL), and the mixture was extracted with ethyl acetate (30 mL×3). The organic phases were combined, washed with saturated brine (10 mL×1), dried over anhydrous sodium sulfate, filtered, and the filtrate was concentrated under reduced pressure. The residue was purified by silica gel column chromatography (eluent: dichloromethane/methanol=100/1 to 10/1) to obtain compound AA. MS-ESI calculated for [M+H]⁺244, found 244.

### Example 2: Preparation of Intermediate BB-5

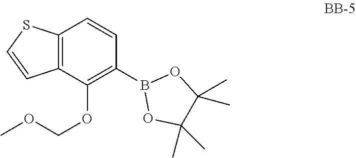

### Synthetic Route:

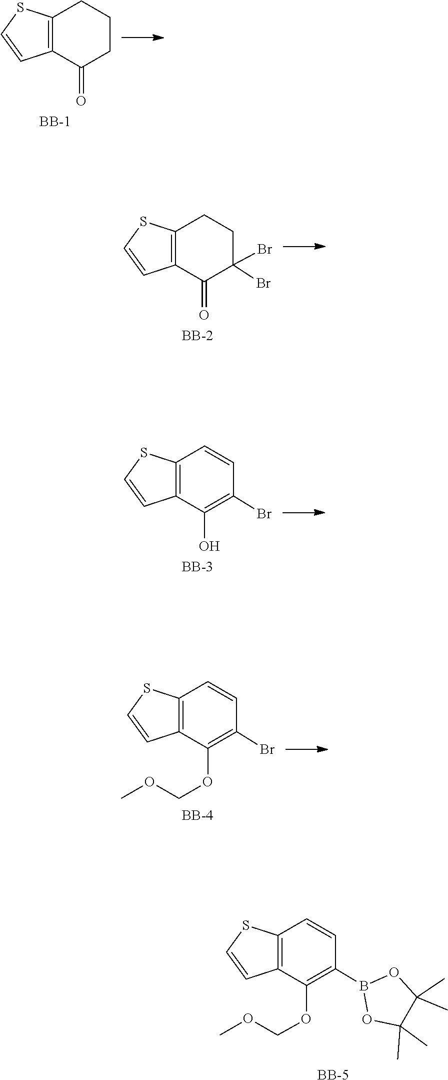

### Step 1

The mixture of copper bromide (733.69 g, 3.28 mol) and ethyl acetate (1200 mL) was stirred at 80° C. for 10 minutes. Compound BB-1 (125 g, 821.22 mmol) was dissolved in chloroform (1200 mL) and then added to the above reaction mixture. The reaction mixture was heated and stirred at 80° C. for 12 hours. After cooling, the reaction mixture was filtered through neutral alumina and diatomite. The filter cake was washed with dichloromethane (1000 mL), and the filtrate was concentrated under reduced pressure to obtain a crude product. The crude product was added with ethyl acetate (500 mL), stirred, filtered, and the filter cake was collected and dried to obtain compound BB-2. ¹H NMR (400 MHz, CDCl₃) δ 7.48 (d, J=5.2 Hz, 1H), 7.18 (d, J=5.2 Hz, 1H), 3.20-3.15 (m, 4H). MS-ESI calculated for [M+H]⁺311, found 311.

### Step 2

Compound BB-2 (215 g, 693.54 mmol) was dissolved in DMF (1000 mL), and lithium carbonate (307.47 g, 4.16 mol) was added to the reaction mixture. The reaction mixture was stirred at 100° C. for 6 hours. The reaction mixture was filtered. The filter cake was washed with DMF (1000 mL), and the filtrate was concentrated under reduced pressure to (600 mL). The residue was added to water (6000 mL), and the pH was adjusted to 1 with dilute hydrochloric acid (1 mol/L). The mixture was stirred at room temperature for 30 minutes, filtered, and the filter cake was collected and dried to obtain compound BB-3. ¹H NMR (400 MHz, CDCl₃) δ 7.51 (d, J=5.6 Hz, 1H), 7.45-7.37 (m, 2H), 7.34 (d, J=8.8 Hz, 1H), 5.87 (s, 1H).

### Step 3

Compound BB-3 (200 g, 873.01 mmol) and potassium carbonate (301.65 g, 2.18 mol) were dissolved in DMF (1000 mL). The reaction mixture was cooled to 0° C. Chloromethyl ether (153.070 g, 1.90 mol) was slowly added dropwise to the reaction mixture. The reaction mixture was reacted at 25° C. for 12 hours. The reaction mixture was added to ice water (5000 mL) under stirring, the pH was adjusted to 10 with saturated potassium carbonate, and the mixture was extracted with ethyl acetate (1000 mL×3). The organic phase was dried over anhydrous sodium sulfate, filtered, and the filtrate was concentrated under reduced pressure. The concentrate was separated by silica gel column chromatography (petroleum ether/ethyl acetate=200/1 to 100/1) to obtain compound BB-4. ¹H NMR (400 MHz, CD₃OD) δ 7.61-7.56 (m, 2H), 7.53-7.46 (m, 2H), 5.24 (s, 2H), 3.66 (s, 3H).

### Step 4

Compound BB-4 (100 g, 366.10 mmol), compound 4,4,5,5-tetramethyl-1,3,2-dioxaborolane (70.28 g, 549.16 mmol), triethylamine (111.14 g, 1.10 mol), palladium acetate (6.58 g, 29.29 mmol), and 2-(dicyclohexylphosphino) biphenyl (20.53 g, 58.58 mmol) were dissolved in dioxane (1000 mL), and the mixture was stirred and reacted at 100° C. for 12 hours under nitrogen atmosphere. The reaction mixture was concentrated under reduced pressure to remove the solvent. The residue was dissolved in ethyl acetate (500 mL), filtered through a silica gel pad, and the filtrate was concentrated under reduced pressure. The concentrate was separated by silica gel column chromatography (petroleum ether/ethyl acetate=200/1 to 100/1) to obtain compound BB-5. ¹H NMR (400 MHz, CD₃OD) δ 7.69-7.65 (m, 2H), 7.57-7.52 (m, 2H), 5.24 (s, 2H), 3.59 (s, 3H), 1.39 (s, 12H).

### Example 3: Preparation of Compound II

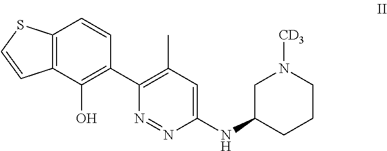 [SMILES: [2H]C([2H])([2H])N1CCC[C@@H](Nc2cc(C)c(-c3ccc4sccc4c3O)nn2)C1]

### Synthetic Route:

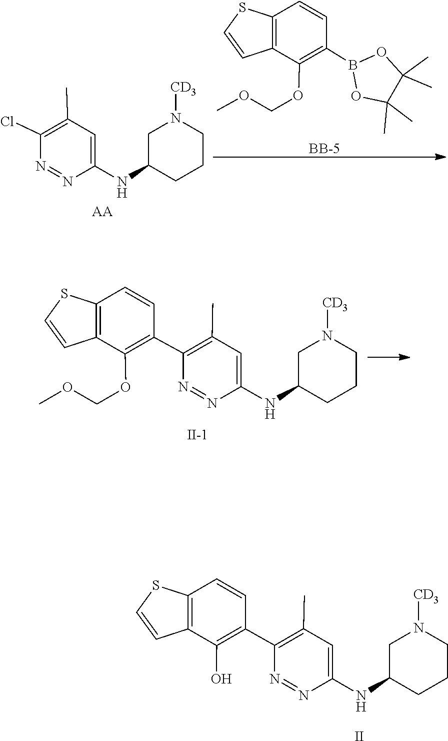

### Step 1

Compound AA (620.00 mg, 2.54 mmol), compound BB-5 (1.06 g, 3.31 mmol), and potassium carbonate (703.10 mg, 5.09 mmol) were dissolved in 1,4-dioxane (10 mL) and water (1 mL). The reaction system was purged with nitrogen, and the reaction mixture was added with [1,1′-bis(diphenylphosphino) ferrocene]dichloropalladium (II) dichloromethane (207.72 mg, 254.36 μmol). The reaction mixture was stirred and reacted at 90° C. for 12 hours under nitrogen atmosphere. The reaction mixture was added with water (10 mL) and extracted with ethyl acetate (20 mL×2). The organic phases were combined, washed with saturated brine (5 ml×1), dried over anhydrous sodium sulfate, filtered, and the filtrate was concentrated under reduced pressure. The residue was purified by silica gel column chromatography (eluent: dichloromethane/methanol=100/1 to 10/1) to obtain compound II-1. ¹H NMR (400 MHz, CD₃OD) δ 7.80 (d, J=8.4 Hz, 1H), 7.63 (d, J=5.6 Hz, 1H), 7.55 (d, J=5.6 Hz, 1H), 7.28 (d, J=8.4 Hz, 1H), 6.81 (s, 1H), 4.94 (s, 2H), 4.23-4.10 (m, 1H), 3.16 (s, 3H), 2.87-2.73 (m, 1H), 2.34-2.16 (m, 2H), 2.10 (s, 3H), 2.10-1.97 (m, 2H), 1.92-1.74 (m, 2H), 1.74-1.58 (m, 1H), 1.50-1.37 (m, 1H). MS-ESI calculated for [M+H]⁺402, found 402.

### Step 2

Compound II-1 (485.10 mg, 1.21 mmol) was dissolved in hydrochloric acid (2 mol/L, 4.83 mL) at 0° C. The reaction mixture was stirred at 25° C. for 3 hours. The pH of the reaction mixture was adjusted to 9 with ammonia water at 0° C. The reaction mixture was prepared and separated by high performance liquid chromatography (chromatographic column: Phenomenex C18 80×40 mm×3 μm; mobile phase: [water (ammonia water+ammonium bicarbonate)−acetonitrile]; acetonitrile: 37% to 67%, 8 minutes) to obtain compound II. ¹H NMR (400 MHz, CDCl₃) δ 7.68 (d, J=5.6 Hz, 1H), 7.44-7.42 (m, 2H), 7.35 (d, J=5.6 Hz, 1H), 6.69 (s, 1H), 5.39-5.30 (m, 1H), 4.25-4.15 (m, 1H), 2.65-2.55 (m, 3H), 2.49 (s, 3H), 2.33-2.30 (m, 1H), 1.85-1.62 (m, 4H). MS-ESI calculated for [M+H]⁺358, found 358.

### Bioactivity Testing:

### Experimental Example 1: IC₅₀ Assay for the Detection of NLRP3 Inhibitors Using THP-1 Cells

Chemical names and structural formulas of the compounds of the present disclosure used in the experiments can be found in the preparation examples of the compounds.

1. Experimental principle: This experiment utilized the human monocytic cell line THP-1 to study the inhibitory activity (IC₅₀) of NLRP3 inhibitors on the secretion of IL-1β by cells. The monocytic cell line THP-1 was differentiated into mature macrophages using PMA (phorbol-12-myristate-13-acetate). The cells were then stimulated with LPS (lipopolysaccharide), a Toll-like receptor 4 (TLR4) agonist, to activate the transcriptional activity of the NLRP3 inflammasome and the expression of pro-IL-1β (the precursor of Il-1β). At this point, NLRP3 inhibitors were added, followed by the addition of ATP (adenosine triphosphate) to further mature and activate NLRP3 and activate downstream caspase-1. Activated caspase-1 could enzymatically cleave pro-IL-1β into mature IL-1β, which could be secreted. NLRP3 inhibitors can effectively inhibit the ATP-induced maturation and activation of NLRP3 and the activation of downstream caspase-1, thereby inhibiting the maturation and secretion of IL-1β.

### 2. Experimental Materials:

### 2.1 Reagents: As Shown in Table 3.

TABLE 3
Experimental reagent details

| | | Cat. No. or | Storage | |
|---|---|---|---|---|
| Name | Supplier | Serial No. | condition | |
| PMA | Sigma | 79346 | −20° | C. |
| LPS | InvivoGen | tlrl-eblps | −20° | C. |
| ATP | | | −20° | C. |
| 1640 culture medium | Gibco | 22400-089 | 4° | C. |
| FBS | HyClone | SV30087.03 | −80° | C. |
| Penicillin-streptomycin | HyClone | SV30010 | 4° | C. |
| β-Mercaptoethanol | Sigma | M3148 | Room | |
| | | | temperature | |
| NEAA Non-essential amino | Gibco | 1140-050 | 4° | C. |
| acids | | | | |

| | | | |
|---|---|---|---|
| Human soluble protein kit | BD | 558265 | Room |
| | | | temperature |
| Human IL-1β Flex Set | BD | 558279 | Room |
| | | | temperature |
| 96-well flat-bottomed plate | Corning | 3599 | Room |
| | | | temperature |
| 96-well U-bottom plate | Corning | 3799 | Room |
| | | | temperature |

### 2.2 Instruments:

Microplate reader, supplier: PerkinElmer, Cat. No. or serial No.: Envision.

### 3. Experimental Steps:

(1) The density of THP-1 cells was adjusted to 5×10⁵ cells/mL, then PMA was added, and the final concentration was adjusted to 100 ng/mL. The cells were seeded into a 96-well flat-bottom plate at 200 μL per well and stimulated overnight at 37° C. with 5% CO₂ (<16 hours if possible).
(2) The next day, the supernatant was discarded, and the cells were carefully washed twice with Dulbecco's phosphate-buffered saline (200 μL per wash).
(3) The cells were stimulated with LPS at a final concentration of 100 ng/mL. LPS was added at 200 μL per well in the 96-well plate and incubated at 37° C. with 5% CO₂ for 3 hours.
(4) The test compounds were added to the wells at screening concentrations of 5 μM, 1 μM, 200 nM, 40 nM, 8 nM, 1.6 nM, 0.32 nM, and 0.064 nM. The plate was incubated in a 37° C., 5% CO₂ incubator for 1 hour.
(5) ATP was added to each well to a final concentration of 5 mM and incubated overnight (>18 hours) at 37° C. with 5% CO₂.
(6) On the third day, 5 μL of the supernatant was taken and diluted 10-fold. The IL-1β content in the supernatant was measured using the Cytometric Bead Array (CBA) technique.

### 4. The Experimental Results are Shown in Table 4:

Table 4 Test results of IC₅₀ assay for the detection of NLRP3 inhibitors using THP-1 cells

| Test compound | IC₅₀ (pM) |
|---|---|
| Compound II | 61.74 |

Conclusion: The compounds of the present disclosure have significant inhibitory activity on the maturation and secretion of IL-1β in THP-1 cells.

### Experimental Example 2: LPS-Stimulated IL-1β Release Assay in Rat Primary Microglia

### I. Experimental Objective:

This experiment utilized rat primary microglia to study the inhibitory activity of NLRP3 inhibitors on the secretion of IL-1β in rat primary microglia.

### II. Experimental Reagents: See Table 5 for Experimental Reagent Details.

TABLE 5
Experimental reagent details

| Reagents and consumables | Supplier | Cat. No. |
|---|---|---|
| DMEM | Gibco | 11995-065 |
| Fetal bovine serum (FBS) | AusGeneX | FBS500-S |
| Penicillin-Streptomycin | Gibco | 15140-122 |
| (Pen/Strep) | | |
| 0.25% Trypsin-EDTA | Gibco | 25200-072 |
| Dulbecco's phosphate buffered | Solarbio | D1040 |
| saline (DPBS) | | |
| HEPES | ChemCruz | sc-29097A |
| Glucose | Sigma | G8270-5KG |
| PDL | Gibco | A38904-01 |
| Trypan blue | Invitrogen | T10282 |
| Dimethyl sulfoxide (DMSO) | Solarbio | D8371 |
| LPS | Sigma | L2880 |
| Nigericin | MCE | HY-100381 |
| MCC950 | MCE | HY-12815A |
| rat IL-1β ELISA Kit | 4A BIOTECH | CRE0006 |
| Cell culture flask (T75) | Corning | 430641 |
| Cell culture plate (96-well) | Corning | 3599 |
| Dilution plate (96-well) | Thermo Scientific | 249944 |

### III. Experimental Operation

Newborn SD rats were used to obtain mixed glial cells from the cerebral cortex through digestion and separation, and the cells were seeded into culture flasks for cultivation. The culture medium was replaced every 3-4 days, and the cells were cultured for approximately 10 days until they reached full confluence. The microglial cells were then collected through shaking at 37° C. and centrifugation, and were seeded into a 96-well cell culture plate for overnight cultivation. The cells were then transferred to serum-free medium and treated with 50 ng/ml LPS for 3 hours. Different concentrations of the compounds were added for 0.5 hours, followed by the addition of 0.3 μg/mL Nigericin for 1 hour. The supernatant was collected and either stored at −80° C. or directly detected for IL-1β release using an ELISA kit, following the manufacturer's instructions. Data processing involved using 10 μM of the positive compound group as the low value (L) and the DMSO group as the high value (H). The inhibition rate of the compound was calculated using the formula: Inhibition %=(Ave_H-Sample)/(Ave_H−Ave_L)*100, and the IC₅₀ of the compound was calculated using the nonlinear regression equation: Y=Bottom+(Top−Bottom)/(1+10∧((LogIC₅₀−X)*HillSlope)).

### IV. The Experimental Results are Shown in Table 6.

TABLE 6
Test results of IC₅₀ assay for the detection
of NLRP3 inhibitors in rat primary microglia

| Test compound | IC₅₀ (nM) |
|---|---|
| MCC950 | 80.20 |
| Compound II | 35.84 |

Conclusion: The compounds of the present disclosure have significant inhibitory activity on the maturation and secretion of IL-1β in rat primary microglia.

### Experimental Example 4: Pharmacokinetic Evaluation

Experimental objective: To test the pharmacokinetics of compound in SD rats in vivo

### Experimental Materials:

SD rat (male, 150 to 180 g, Beijing Marshall BioResources Co., Ltd.)

### Experimental Operation:

The pharmacokinetic characteristics of the compound in rodents after intravenous injection and oral administration were tested according to the standard protocol. In the experiment, the candidate compound was formulated into clear solutions, and a single intravenous injection and oral administration were given to SD rats. The vehicle for intravenous injection and oral administration was a mixed vehicle composed of 5% dimethyl sulfoxide and 95% solutol with a content of 5%. Four male SD rats were used in this project. Two SD rats were administered via intravenous injections at a dose of 2 mg/kg, and plasma samples were collected at 0.083, 0.25, 0.5, 1, 2, 4, 8, and 24 h post-administration. The other two SD rats were administered orally by gavage at a dose of 10 mg/kg, and plasma samples were collected at 0.25, 0.5, 1, 2, 4, 8, and 24 h post-administration. Plasma concentrations were quantitatively analyzed by LC-MS/MS analysis method, and pharmacokinetic parameters such as peak concentration (Cₘₐₓ), clearance (CL), half-life (T_{1/2}), volume of distribution at steady state (Vdss), area under the drug-time curve (AUC₀₋ₗₐₛₜ), and bioavailability (F) were calculated.

TABLE 7
Pharmacokinetic test results

| | | | | | Area | Area | |
|---|---|---|---|---|---|---|---|
| | | | | | under | under | |
| | | | | | drug- | drug- | |
| | | | Volume of | Half- | time | time | |
| | | Clearance | distribution | life | curve | curve | |
| | Peak | CL | at steady | T_{1/2} | AUC₀₋ₗₐₛₜ | AUC₀₋ₗₐₛₜ | Bio- |
| | concentration | (mL/min/ | state Vdss | (IV, | IV | PO | availability |
| Compound | Cₘₐₓ (nM) | kg) | (L/kg) | h) | (nM · hr) | (nM · hr) | F (%) |
| Compound | 593 | 47.4 | 6.99 | 1.82 | 1873 | 2354 | 27.1 |
| II | | | | | | | |

Conclusion: The compound II of the present disclosure has good pharmacokinetic properties in SD rats in vivo, including good oral bioavailability, oral exposure, half-life, clearance, etc.

### Experimental Example 5: Pharmacokinetic Evaluation

Experimental objective: To test the pharmacokinetic of compound in beagle dogs in vivo

### Experimental Materials:

Beagle dogs (male, 5 to 15 kg, Beijing Marshall BioResources Co., Ltd.)

### Experimental Operation:

The pharmacokinetic characteristics of the compound in beagle dogs after oral administration were tested according to the standard protocol. In the experiment, the candidate compound was formulated into clear solutions, and a single intravenous injection and oral administration were given to beagle dogs. The vehicle for intravenous injection and oral administration was a mixed vehicle composed of 5% dimethyl sulfoxide and 95% solutol with a content of 5%. Four male beagle dogs were used in this project. Two beagle dogs were administered via intravenous injections at a dose of 1 mg/kg, and plasma samples were collected at 0.083, 0.25, 0.5, 1, 2, 4, 8, and 24 h post-administration. The other two beagle dogs were administered orally by gavage at a dose of 2 mg/kg, and plasma samples were collected at 0.25, 0.5, 1, 2, 4, 8, and 24 h post-administration. Plasma concentrations were quantitatively analyzed by LC-MS/MS analysis method, and pharmacokinetic parameters such as peak concentration (Cₘₐₓ), clearance (CL), half-life (T_{1/2}), volume of distribution at steady state (Vdss), area under the drug-time curve (AUC₀₋ₗₐₛₜ), and bioavailability (F) were calculated. The experimental results are shown in Table 8:

TABLE 8
Pharmacokinetic test results

| | | | | | Area | Area | |
|---|---|---|---|---|---|---|---|
| | | | | | under | under | |
| | | | | | drug- | drug- | |
| | | | Volume of | Half- | time | time | |
| | | Clearance | distribution | life | curve | curve | |
| | Peak | CL | at steady | T_{1/2} | AUC₀₋ₗₐₛₜ | AUC₀₋ₗₐₛₜ | Bio- |
| | concentration | (mL/min/ | state Vdss | (IV, | IV | PO | availability |
| Compound | Cₘₐₓ (nM) | kg) | (L/kg) | h) | (nM · hr) | (nM · hr) | F (%) |
| Compound | 504 | 25.7 | 6.9 | 3.53 | 1919 | 3106 | 80.9 |
| II | | | | | | | |

Conclusion: The compound II of the present disclosure has good pharmacokinetic properties in beagle dogs in vivo, including good oral bioavailability, oral exposure, half-life, clearance, etc.

1. A compound of the following formula, a stereoisomer thereof, or a pharmaceutically acceptable salt thereof,

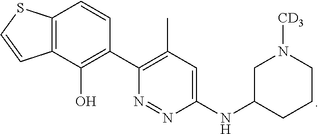 [SMILES: [2H]C([2H])([2H])N1CCCC(Nc2cc(C)c(-c3ccc4sccc4c3O)nn2)C1]

2. The compound, the stereoisomer thereof, or the pharmaceutically acceptable salt thereof according to claim 1, wherein the compound is selected from:

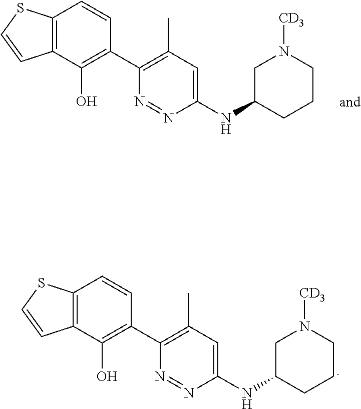 [SMILES: [2H]C([2H])([2H])N1CCC[C@@H](Nc2cc(C)c(-c3ccc4sccc4c3O)nn2)C1 ; [2H]C([2H])([2H])N1CCC[C@H](Nc2cc(C)c(-c3ccc4sccc4c3O)nn2)C1]

3. (canceled)

4. A method for treating Parkinson's disease in a subject in need thereof, comprising administering to the subject an effective amount of the compound, the stereoisomer thereof, or the pharmaceutically acceptable salt thereof according to claim 1.

5. A method for treating Parkinson's disease in a subject in need thereof, comprising administering to the subject an effective amount of the compound, the stereoisomer thereof, or the pharmaceutically acceptable salt thereof according to claim 2.

6. An NLRP3 inhibitor, comprising the compound, the stereoisomer thereof, or the pharmaceutically acceptable salt thereof according to claim 1.

7. An NLRP3 inhibitor, comprising the compound, the stereoisomer thereof, or the pharmaceutically acceptable salt thereof according to claim 2.
